(19) United States
(12) Patent Application Publication
(10) Pub. No.: US 2007/0059300 A1
(43) Pub. Date: Mar. 15, 2007

(54) COMPOUNDS AND COMPOSITIONS TO CONTROL ABNORMAL CELL GROWTH

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: ZOLTAN LABORATORIES LLC, Austin, MN (US)

(21) Appl. No.: 11/461,916

(22) Filed: Aug. 2, 2006

Related U.S. Application Data
(60) Provisional application No. 60/716,346, filed on Sep. 12, 2005.
(60) Provisional application No. 60/732,165, filed on Oct. 31, 2005.

(51) Int. Cl. A61K 38/46; A61K 38/40

(52) U.S. Cl. 424/94.6; 514/6

(57) ABSTRACT

Methods and regimens for stimulating proliferation and inhibiting death of skin cells in wounded, non-wounded healthy and unhealthy, or transplanted skin and skin cell suspensions are provided. The methods and regimens include a step of administering to an area of host skin and/or transplanted skin a combination of lactoferrin and alkaline phosphatase such as placental alkaline phosphatase. The active components may be administered topically, by injection, or by other suitable means. In addition to improving the quality of skin, the reported methods may also be effective to reduce inflammation and microbial infection in the skin. Embodiments of the invention also provide compositions suitable for use in the described methods.

### REFERENCE TO RELATED APPLICATIONS

This application claims the benefit of U.S. provisional patent application Ser. No. 60/732,165 filed Oct. 31, 2005 and entitled “PROTEIN COMPOSITION FOR HEALING SKIN INJURY AND DAMAGE” which is hereby incorporated by reference in its entirety.

### TECHNICAL FIELD

Embodiments of the present invention relate to the promotion of wound healing as well as prevention and repair of stress-induced unfavorable changes in skin's morphology and function via administration of agents that may help enhance survival and proliferation of skin cells and control inflammation and skin infection.

### BACKGROUND

There is an unmet need in the health care sector for products that would effectively prevent stress-induced deterioration of skin's structure and function and promote healing of skin damage or wounding caused by major surgeries, accidents, cosmetic procedures, sunlight, aging, or a medical condition including diabetes and poor circulation. The absence of effective wound healing products on the market can be explained by the very complex nature of the healing process, which is difficult to influence externally without risking of introducing adverse effects. The situation may be further complicated when the subject also has a medical condition, such as diabetes or poor circulation, which inhibits the natural course of healing.

Normally, repair of non-chronic wounds involves three major partially overlapping complex processes that follow a specific time sequence: inflammation, tissue formation, and tissue remodeling [Martin, P. (1997), “Wound healing-Aiming for perfect skin regeneration,” Science 276, 75-81]. Numerous growth factors and cytokines regulate these events in a coordinated manner [Werner S. and Grose, R. (2003), “Regulation of wound healing by growth factors and cytokines,” Physiol. Rev. 10, 835-870]. Because of the complexity of these processes, application of a single growth factor does not seem to be sufficient to significantly promote wound healing. For example, platelet-derived growth factor (sold under the name of “REGENEREX”) was approved by FDA for wound healing application, but proved relatively ineffective in clinical practice. A possible reason for this is that platelet-derived growth factor stimulates only the proliferation of fibroblasts in the deeper dermis layer, and this may not be sufficient to significantly impact the proliferation of keratinocytes (epidermal cells in the epidermis).

Another possibility to promote wound healing could be application of a mixture or combination of growth factors. However, most growth factors are short-lived (due to rapid degradation by proteases present in the wound fluid) and often counteract each other's effects, making it difficult to establish an optimal dose. For example, transforming growth factor-α and transforming growth factor-β have stimulatory and inhibitory effects on keratinocyte proliferation, respectively [Werner S. and Grose, R. (2003), “Regulation of wound healing by growth factors and cytokines,” Physiol. Rev. 10, 835-870]. High-dose epidermal growth factor induces the production of transforming growth factor-β and thereby inhibits proliferation of keratinocytes [Yamasaki, K., Toriu, N., Hanakawa, Y., Shirakata, Y., Sayama, K., Takayanagi, A., Ohtsubo, M., Gamou, S., Shimizu, N., Fujii, M., Miyazono, K, and Hashimoto, K. (2003), “Keratinocyte growth inhibition by high-dose epidermal growth factor is mediated by transforming growth factor-β autoinduction: A negative feedback mechanism for keratinocyte growth,” J. Invest. Dermatol. 120, 1030-1037]. In other cases, lower dose of epidermal growth factor was found to promote wound healing [Werner S. and Grose, R. (2003), “Regulation of wound healing by growth factors and cytokines,” Physiol. Rev. 10, 835-870]. Because of its contradictory effects at different concentrations, it is very difficult to establish an effective dose for epidermal growth factor. For these and other reasons, to the best knowledge of this inventor development of an effective combination of growth factors to promote wound healing has remained elusive. There has been a similar lag in the development of non-growth factor promoters of wound healing.

In adults, inflammation elicits platelet degranulation as well as infiltration of monocytes and macrophages into the wound site associated by the release of growth factors and cytokines that promote healing. There is a general view that in adults such inflammatory response, which eventually results in scar formation, is excessive. In contrast, fetal wound healing does not elicit a major inflammatory response, and, as a result it occurs without significant scarring. Transforming growth factor-β is an endogenous protein which is highly expressed in adult wounds, but not in fetal wounds, and which is generally accepted to be a major promoter of scar tissue formation. Based on these observations, a recent trend among dermatologists is to attempt to reduce the inflammation phase to avoid excessive scarring during healing, and externally replace only those growth factors that promote healing but not scarring.

Healing of chronic wounds, such as various ulcers, is further complicated by several factors usually including continuous inflammation and decreased formation of growth factors resulting in the inhibition of tissue formation and tissue remodeling. Chronic inflammation can be triggered by obesity that is often associated with high blood glucose level and diabetes. As in case of non-chronic wounds, control of inflammation is an important step toward optimum healing of chronic wounds. Therefore, an agent or a combination or mixture of agents that both reduce inflammation and promote survival and proliferation of skin cells may be suitable for facilitating healing of both non-chronic and chronic wounds as well as promoting the survival of skin transplants and the regeneration of damaged skin.

Intact skin transplants or skin cell suspensions are often used to cover larger areas of wounded skin to provide protection and facilitate regeneration of one's own skin. The success of infection-free skin transplantation depends on how fast the incorporation of transplanted skin into the host and regeneration of own skin tissue occurs. Thus, there is interest to apply agents that can make the transplantation process more efficient by enhancing survival and proliferation of both the transplanted and host skin cells.

### SUMMARY OF THE INVENTION

In one embodiment, the present invention includes a composition comprising a physiologically compatible carrier and a therapeutically effective amount of a combination including lactoferrin (also called lactotransferrin) or active derivative thereof, and alkaline phosphatase or an active derivative thereof. The alkaline phosphatase may include placental alkaline phosphatase, intestinal alkaline phosphatase, tissue non-specific alkaline phosphatase, or germ-cell alkaline phosphatase, or their active derivatives. Upon administration, the combination may be effective to promote survival and proliferation of skin cells in the epidermis and dermis of wounded as well as healthy and unhealthy skin. The combination may also be effective to reduce inflammation and infection in the epidermis and dermis of a subject's skin.

The combination described herein is demonstrated in the Examples below to enhance healing of burn wounds. It is expected that administration of the combination is also effective to enhance the healing of other kinds of wounds including cuts and chronic wounds (such as diabetic ulcer) as well as promote survival of skin transplants and regeneration of damaged skin. Embodiments of the present invention may be suitable in the treatment of wounds or healthy and unhealthy (but non-wounded) skin as well as transplanted skin by the simultaneous or sequential administration of a combination of lactoferrin and alkaline phosphatase, or active derivatives thereof. Embodiments of the invention provide methods and regimens that may be suitable for the treatment of wounds, healthy and unhealthy skin, transplanted intact skin, or transplanted skin cell suspension.

In view of the complex nature of the skin repair process, significant effects on healing of wounds with less scar tissue formation and regeneration of damaged skin is likely to require simultaneous application of at least two agents that can control inflammation and microbial load and enhance proliferation of skin cells. In embodiments of the present invention, two proteins, each having a different spectrum of effects, are used to achieve these goals.

One of the two proteins is lactoferrin or lactotransferrin (“Lf”), which is known to exert anti-inflammatory and anti-microbial effects and, as demonstrated here, also exerts positive effects on skin cell survival and proliferation. The other protein is alkaline phosphatase (“PALP”), which is thought to promote survival and renewal of skin cells in wounded as well as healthy and unhealthy skin. In embodiments of the present application, PALP, or its active derivative, is used together with Lf, or its active derivative, both proteins exerting a different spectrum of actions that are expected to promote healing of wounded or damaged skin as well as prevent and reverse stress-induced deterioration of skin.

Embodiments of the invention also provide methods and regimens involving administration or application of the combination described herein. In one embodiment the application is topical. In another embodiment the administration is intradermal. In yet another embodiment of the method the administration is by intravenous, subcutaneous, intramuscular, or intraperitoneal injection. In further embodiments, topical application and administration by injection can be used simultaneously or sequentially. In additional embodiments, the combination can be added to skin cell suspension prior to transplantation and/or directly to the skin area populated by newly transplanted skin cells.

In one embodiment, the invention provides a method for promoting proliferation or survival of skin cells in the epidermis and dermis of wounded skin or in healthy or unhealthy skin of a subject, comprising the step of topically administering a composition to the target skin area. Embodiments of the invention further include a method for promoting proliferation or survival of skin cells in the epidermis and dermis of wounded skin or in healthy or unhealthy skin, comprising the step of injecting a suitable composition.

Yet another embodiment provides a regimen for promoting proliferation or survival of skin cells in the epidermis and dermis of wounded skin or in healthy or unhealthy skin, the regimen comprising periodically administering the combination to the skin either topically or by one of the injection methods.

Embodiments of the invention further provide a method for stimulating proliferation and reducing death of cells of transplanted skin, including a step of topically administering to an area of the transplanted skin therapeutically effective amounts of Lf and PALP, or their active derivatives, dissolved or dispersed in physiologically compatible carriers. In one embodiment the transplanted skin is human skin. In another embodiment of the method, the transplanted skin has been transplanted onto the human host. Another embodiment of the method includes a step of topically administering therapeutically effective amounts of proteins to an area of host skin that is adjacent to the transplanted skin. Yet another embodiment of the method includes a step of topically administering therapeutically effective amounts of proteins both to an area of host skin adjacent to the transplanted skin and directly to the transplanted skin.

Embodiments of the invention also include a method for stimulating proliferation and reducing death of cells of transplanted skin, including the step of injecting the Lf and PALP proteins or their active derivatives by one of the injection methods including injection near to an area of the transplanted skin. Therapeutically effective amounts of Lf and PALP, or their active derivatives, are dissolved or dispersed in a physiologically acceptable carrier. Physiologically acceptable carrier may be, for example, 0.9 M sodium chloride (physiological saline).

Embodiments of the invention also provide a method for stimulating proliferation and reducing death of cells of both the host and transplanted skin by combining topical applications and injection methods to administer the active composition(s) to or near the affected areas of host and transplanted skin.

Still other embodiments of the invention provide a method for stimulating proliferation and reducing death of suspended skin cells prior to transplantation by adding to such suspensions therapeutically effective amounts of Lf and PALP, or their active derivatives. Such treatment may or may not be followed by additional topical treatments at the site of transplantation after stabilization of transplanted cells in the host. In both cases, Lf and PALP may also be applied by one of the injection methods used in the medical practice.

### BRIEF DESCRIPTION OF THE FIGURES

FIG. 1 is a digital image of a histological sample taken from transplanted human skin six days after immersing the skin into 65° C. water and treating twice with vaselinum cholesterinatum alone. Segments of skin are separated from each other and show no sign of any significant regeneration of the epidermis.

FIG. 2 is a digital image of a histological sample taken from transplanted human skin six days after immersing the skin into 65° C. water and treating twice with vaselinum cholesterinatum alone. The dermis is a-cellular and shows no sign of any significant regeneration of the epidermis.

FIG. 3 is a digital image of a histological sample taken from transplanted human skin six days after immersing the skin into 65° C. water and treating twice with human Lf dispersed in vaselinum cholesterinatum. While the density of cells in the dermis is high, there is no clear indication of new epidermis formation in this skin section.

FIG. 4 is a digital image of a histological sample taken from transplanted human skin six days after immersing the skin into 65° C. water and treating twice with human Lf dispersed in vaselinum cholesterinatum. In this skin section, a low-level regeneration of epidermis is seen (indicated by the blue color and the columnar structure of newly forming epidermis in the upper-center part of the picture).

FIG. 5 is a digital image of a histological sample taken from transplanted human skin six days after immersing the skin into 65° C. water and treating twice with human PALP dispersed in vaselinum cholesterinatum. In this skin section, a medium-level regeneration of epidermis is seen (indicated by the blue color and the well-developed columnar structure of newly forming epidermis in the upper part of the picture).

FIG. 6 is a digital image of a histological sample taken from transplanted human skin six days after immersing the skin into 65° C. water and treating twice with human PALP dispersed in vaselinum cholesterinatum. In this skin section, a medium-level regeneration of epidermis is seen represented by two to three layers of newly formed epidermal cells.

FIG. 7 is a digital image of a histological sample taken from transplanted human skin six days after immersing the skin into 65° C. water and treating twice with a mixture of human Lf and human PALP dispersed in vaselinum cholesterinatum. In this skin section, a higher level regeneration of epidermis is seen represented on average by five layers of epidermal cells.

FIG. 8 is a digital image of a histological sample taken from transplanted human skin six days after immersing the skin into 65° C. water and treating twice with a mixture of human Lf and human PALP dispersed in vaselinum cholesterinatum. In this skin section, a higher level regeneration of epidermis is seen represented on average by four to six layers of epidermal cells.

FIG. 9 is a digital image of a histological sample taken from transplanted human skin six days after immersing the skin into 65° C. water and treating twice with a mixture of human Lf and human PALP dispersed in vaselinum cholesterinatum. In this skin section, a higher level regeneration of epidermis is indicated by the reconstructed columnar structure of epidermis.

FIG. 10 is an image of a gel separation demonstrating that the PALP used for the experiments described in Examples 3 and 4 was homogeneous or near homogeneous.

### DETAILED DESCRIPTION

Definitions

As used herein, and unless otherwise specified or established by the context, the terms and phrases set forth below shall have the following meanings:

The term “wound” is used to describe an area of skin where either layers of the epidermis, the whole epidermis, or all skin layers have lost continuity, either morphologically or functionally or both. For example, upon moderate burning, patches of epidermis may be damaged without visible signs of loss of continuity; nevertheless, such epidermis has lost functional continuity so that such area of skin must be regarded as wounded. The term “wound” includes, for example, injuries such as cut, puncture, abrasion, sore, scar, bruise, and burns of moderate- to high-degree.

When used with respect to skin, the terms “damaged” and “unhealthy” refer to a skin area which is morphologically and functionally still intact, but which has a reduced capacity to function normally. For example, in aging skin or in skin exposed to excessive sunlight, the various skin layers, particularly the dermis, contain fewer cells including fibroblasts, and other skin constituents such as collagen. Damaged and unhealthy skin is weaker, less elastic, and is more prone to injury than healthy skin. For the purposes of the present application, skin that is transplanted onto a host is considered to be “damaged” or “unhealthy” until the transplanted skin area completely regains the original function. As used in this application “transplanted skin” may be a transplanted from one to oneself, an autograft; a transplant of skin from a genetically non-identical member of the same species, an allograft; a transplant of skin from one genetically identical other (such as an identical twin), an isograft; or a transplant from one species to another, a xenograft.

When used with respect to a protein preparation, the phrase “highly purified” refers to a preparation where the protein of interest represents 90% or more (by mass) of the total protein content as determined by an SDS-PAGE gel electrophoresis procedure. By way of example, commercial human Lf used in the experiments described herein represents a highly purified protein preparation.

When used with respect to a protein preparation, the phrase “homogeneous” refers to a protein preparation where the protein of interest is the only protein that can be clearly detected (such as by using coomassie blue or silver stain for protein staining) by SDS-PAGE gel electrophoresis. This definition allows for the detection of multiple bands represented by various forms of the same protein (e.g., glycosylated or phosphorylated forms), so long as the proteins in the separate bands have the same amino acid composition. By way of example, a homogeneous PALP preparation used in the experiments described herein contained only one band by SDS-PAGE gel electrophoresis.

The terms “lactoferrin” and “Lf” refer generally to the full-length lactoferrin (also called lactotransferrin) protein, including isoforms and any chemically modified versions such as created by glycosylation, phosphorylation, or iron-binding.

The phrase “active derivative of Lf” is intended to include any segment or region of Lf that alone or in conjunction with PALP (or an active derivative thereof) promotes healing of wounds, at least partially restores the function of wounded or damaged skin, and at least partially prevents stress-induced deterioration of skin. This definition is considered to include any such segment or region that is unmodified or modified by glycosylation, phosphorylation or iron-binding.

The term “PALP” refers generally to the full-length human alkaline phosphatase protein, including isoforms and any chemically modified versions such as created, for example, by glycosylation, phosphorylation, metal-binding, or ligand-binding. As used herein, PALP is not limited to only placental alkaline phosphatase, but also includes intestinal alkaline phosphatase, germ cell alkaline phosphatase, and tissue nonspecific alkaline phosphatase (found in bone, liver, and kidney). This is done in order to make this application more readable to those in the art.

The phrase “active derivative of PALP” is intended to include any segment or region of PALP that alone or in conjunction with Lf promotes healing of wounds, at least partially restores the function of wounded or damaged or unhealthy skin, and at least partially prevents stress-induced deterioration of skin. This definition is considered to include any such segment or region that is unmodified or modified by glycosylation, phosphorylation, metal-binding, or ligand-binding. The term “administered” generally refers to administering the composition of lactoferrin or PALP or both as topical compositions or injectable compositions. The term “sequential” or “simultaneously” refers to the administration of lactoferrin or PALP simultaneously or sequentially but as separate compositions.

Active Components

Lactoferrin

One active component in the methods and compositions of embodiments of the present invention is lactoferrin (Lf), an iron-binding glycoprotein with an approximate molecular weight of 80 kDa. The protein belongs to the transferrin family of iron transporters; however, it binds iron about 250-fold stronger than transferrin does. Lf is abundant in colostrum and smaller amounts also occur in the saliva, tears, and mucous secretions. Lf is also produced by neutrophils and is released by these cells in response to inflammatory stimuli.

Due to its strong affinity for iron, Lf is thought to play a role in iron uptake by the intestinal mucosa of the suckling neonate. Also, binding of iron also accounts for its anti-microbial activity because the growth of bacteria depends on iron. In addition, Lf is thought to have other activities. For example, by binding free iron at sites of inflammation, Lf can prevent oxygen free-radical-induced damage of tissues. Based on this effect, Lf is used as an experimental drug for local treatment of inflammatory diseases [Trif, M., Guillen, C., Vaughan, J. M., Brewer, J. M., Roseanu, A. and Brock, J. H. (2001), “Liposomes as possible carriers for lactoferrin in the local treatment of inflammatory diseases,” Exp. Biol. Med. 226, 559-564].

Wound healing requires formation of new blood vessels, and Lf has been reported to enhance angiogenesis [Norrby, K (2004), “Human apo-lactoferrin enhances angiogenesis mediated by vascular endothelial growth factor A in vivo,” J. Vasc. Res. 41, 293-304].

Bovine Lf was shown to prevent cancer [Tsuda, H., Sekine, K., Fujita, K.-i. and Iigo, M. (2002), “Cancer prevention by bovine lactoferrin and underlying mechanisms: A review of experimental and clinical studies,” Biochem. Cell Biol. 80, 131-136]. Biochemical similarities between carcinogenesis and the wound-healing process have been identified by researchers.

Because of its known properties as well as long half-life time (5-7 days), Lf is a promising candidate as an active agent for wound healing. Lf has already been proposed for the facilitation of wound healing in various patent applications and issued patents. In none of the publications cited below, however, was Lf employed in conjunction with placental alkaline phosphatase.

In U.S. Pat. No. 5,561,109 (issued Oct. 1, 1996, and titled “Method for the healing of wounds caused by corneal injury”) Mita et al. describe the combined use of Lf and lactoperoxidase to heal corneal injury. In U.S. Pat. No. 5,556,645 (issued Sep. 17, 1996 and titled “Methods for enhancing wound healing and tissue repair”), Bockman et al. describe the use of Lf, as a minor component, together with gallium-containing compounds as the main components, to a variety of applications in wound healing including dermatological and cosmetic skin repair, bone fracture repair, bonding of implanted tissue grafts as well as connective and support tissue prostheses. In U.S. Pat. No. 5,686,116 (issued Nov. 11, 1997, titled “Methods of enhancing repair, healing and augmentation of bone implants”), Bockman et al. describe the use of Lf, as a minor component, together with group IIIa element-containing compounds to induce cells responsible for repair, healing and augmentation of tissues and organs. In a U.S. Pub. App. 2004/0142037 (filed Sep. 16, 2003, titled “Lactoferrin compositions and methods of wound treatment”), Engelmayer et al. describe lactoferrin-containing compositions to treat wounds. In U.S. Pub. App. 2003/0133989 (filed Oct. 1, 2002, titled “Skin care products containing whole egg”), Marenick describes Lf, as a minor component, for the use of wound healing products that comprise whole egg as the major component. In none of these issued and submitted patent applications was placental alkaline phosphatase mentioned as being used alone or in combination with Lf for promoting wound healing.

Finally, in U.S. Pub. App. 2003/0109422 (filed Aug. 14, 2002, titled “Process for the preparation of pharmaceutical formulations containing lactoferrin”), Mazzone et al. describe a process for the preparation of pharmaceutical formulations containing lactoferrin. None of the formulations presented in this application included placental alkaline phosphatase.

Lf is also an interesting protein in that smaller peptides derived from the whole protein are often biologically active. As an example, lactoferricin B 4-14, an 11 amino acid peptide that is derived from bovine lactoferrin, is commercially available. This peptide (Sigma-Aldrich catalog number: L 1290) protects mucosal surfaces from bacterial infection. Several other lactoferricin B (bovine) and lactoferricin H (human) analogs with similar antimicrobial activities have been described as listed by Vogel et al. [Vogel, H. J., Schibli, D. J., Jing, W., Lohmeier-Vogel, E. M., Epand, R. F. and Epand, R. M. (2002), “Towards a structure-function analysis of bovine lactoferrin and related tryptophan- and arginine-containing peptides,” Biochem. Cell Biol. 80, 49-63]. Another peptide derived from Lf was shown to induce death of leukemic cells [Roy, M. K., Kuwabara, Y., Hara, K., Watanabe, Y. and Tamai, Y. (2002), “Peptides from the N-terminal end of bovine lactoferrin induce apoptosis in human leukemic (HL-60) cells,” J. Dairy Sci. 85, 2065-2074]. Separate regions of the lactoferrin and lactoferricin molecules may mediate the anti-microbial, anti-fungal, anti-tumor and anti-viral effects as well as the anti-inflammatory and immunomodulatory effects, respectively [Vogel, H. J., Schibli, D. J., Jing, W., Lohmeier-Vogel, E. M., Epand, R. F. and Epand, R. M. (2002), “Towards a structure-function analysis of bovine lactoferrin and related tryptophan- and arginine-containing peptides,” Biochem. Cell Biol. 80, 49-63].

Accordingly, any segment or region of Lf having not more amino acid units than the full-length Lf and unmodified or modified by glycosylation, phosphorylation or iron binding, that promotes healing of wounds and/or restoration of function of damaged skin alone and/or in conjunction with PALP is considered to be an active derivative of Lf, and is suitable for the practice of embodiments of the invention. Relatively minor alterations in the structure of Lf or its active derivatives, such as exchanging one or more amino acids at critical positions, may enhance the biological activities of the resulting protein as related to the promotion of healing of wounded or damaged skin. The use of such altered or modified proteins is within the scope of embodiments of the present invention as much as the alterations or modifications remain limited and result in an active derivative that is a biologically functional equivalent. “Biologically functional equivalent” in this usage means that any changes result in a molecule that has similar or enhanced biological activity.

Such modified or altered versions of Lf or its derivatives can be produced chemically or by known methods of mutagenesis, for example site-directed mutagenesis. The recombinant forms of the modified peptides can be expressed and produced in any of the suitable cell lines or organisms as described above.

One consideration in the practice of embodiments of the invention is the degree of purity of Lf or its active derivative that is required for skin treatment. Using a preparation that comprises highly purified (at least 90% pure) Lf in the methods and treatment regimens of embodiments of the present invention has the advantage that possible side effects caused by contaminating proteins will probably be negligible. However, impure Lf (or its active derivative) also can be used in the compositions described herein, as long as no adverse effects are observed.

Various forms of Lf may be suitable for use in embodiments of the invention. Human Lf is suitable for the practice of embodiments of the invention, for example. Chromatographically highly purified human Lf, essentially containing only one major band by SDS-PAGE gel electrophoresis, is available from Sigma Aldrich (catalog no. L 0520); this preparation was used for experiments described in the Examples below.

Purified recombinant human Lf, which is biologically as effective as endogenous Lf [Dial, E. J., Dohrman, A. J., Romero, J. J. and Lichtenberger, L. M. (2005), “Recombinant human lactoferrin prevents NSAID-induced intestinal bleeding in rodents,” J. Pharm. Pharmacol. 57, 93-99, and other relevant articles referenced in this article], can be purchased from Agennix Inc. (Houston, Tex.). Bacteria, yeast, fungi, mammalian cells, and various transgenic plants have all been used to produce recombinant human Lf. Also, attempts are being made to produce recombinant human Lf in genetically modified cows wherein Lf could be extracted from milk. The biologically active N-terminal peptide can be obtained through digestion with the protease pepsin A as described by others [Roy, M. K., Kuwabara, Y., Hara, K., Watanabe, Y. and Tamai, Y. (2002), “Peptides from the N-terminal end of bovine lactoferrin induce apoptosis in human leukemic (HL-60) cells,” J. Dairy Sci. 85, 2065-2074]. Lf-derived peptides containing up to about 25 amino acids can also be chemically synthesized by conventional solid-phase synthesis techniques.

Alkaline Phosphatase

The second active component in the methods and compositions of embodiments of the present invention is human alkaline phosphatase, or an active derivative thereof. Out of placental alkaline phosphatase, intestinal alkaline phosphatase, germ cell alkaline phosphatase, and tissue nonspecific alkaline phosphatase, only placental alkaline phosphatase stimulated fibroblast growth in vitro in a commercial cell culture medium (Dulbecco's Modified Eagle's medium; Sigma-Aldrich Company). At present it is not clear if stimulation of wound healing in vivo is entirely or only partly mediated via direct stimulation of skin cell proliferation. Some indirect mechanisms requiring alkaline phosphatase activity, for example local stimulation of growth factor production by fibroblasts or infiltrating immune cells may significantly contribute to the stimulatory effect of PALP on wound healing in vivo.

Also, most recently this inventor found that in vitro, placental alkaline phosphatase, intestinal alkaline phosphatase, and tissue nonspecific alkaline phosphatase had similar (6 to 8-fold) stimulatory effects on DNA synthesis in embryonal mouse NIH 3T3 fibroblasts when 2 mM calcium chloride (final concentration) was added to the cell culture medium. It is important to note that in the wounded epidermis the extracellular concentration of calcium can easily reach 2 mM or even higher concentration (instead of the more physiological 1.1 mM). For these reasons, it is reasonable to expect that other alkaline phosphatases may at least partly mimic the stimulatory effects of placental alkaline phosphatase on wound healing in combination with Lf.

PALP is a member of the alkaline phosphatase group of enzymes that hydrolyze phosphate-containing compounds at alkaline pH. In humans, alkaline phosphatases are encoded by four distinct loci. Three isozymes, including the intestinal alkaline phosphatase, placental alkaline phosphatase, and germ cell alkaline phosphatase are tissue specific. Also, they are 90-98% homologous. The fourth enzyme is tissue nonspecific alkaline phosphatase that can be found in bone, liver and kidney. Tissue nonspecific alkaline phosphatase is about 50% identical with the other three enzymes [J. L. Millan, and W. H. Fishman (1995), “Biology of human alkaline phosphatases with special reference to cancer,” Critical Reviews in Clinical Sciences 32, 1-39]. Placental alkaline phosphatase, in contrast to the other three alkaline phosphatases, is present only in humans and apes. Mature PALP is a dimer of two identical glycosylated subunits. Each subunit has an approximate molecular weight of 66 kDa, as determined by gel electrophoresis.

The present inventor earlier reported that PALP can enhance both the proliferation [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167] and survival [Q.-B. She, J. J. Mukherjee, T. Chung, and Z. Kiss (2000), “Placental alkaline phosphatase, insulin, and adenine nucleotides or adenosine synergistically promote long-term survival of serum-starved mouse embryo and human fetus fibroblasts,” Cellular Signalling 12, 659-665] of mouse embryo fibroblasts as well as fibroblast-like cells derived from the lung of human fetus.

While placental alkaline phosphatase significantly protects cells against certain death-inducing environmental effects, such as nutrient deprivation-associated oxygen free radicals [Q.-B. She, J. J. Mukherjee, T. Chung, and Z. Kiss (2000), “Placental alkaline phosphatase, insulin, and adenine nucleotides or adenosine synergistically promote long-term survival of serum-starved mouse embryo and human fetus fibroblasts,” Cellular Signalling 12, 659-665], it is not known to exert anti-inflammatory and anti-microbial effects.

In a recently issued U.S. patent [U.S. Pat. No. 7,011,965, entitled “Compositions and Methods for Stimulating Wound Healing and Fibroblast Proliferation”] and a recent U.S. patent application [Sep. 2, 2003 and entitled “Use of Placental Alkaline Phosphatase to Promote Skin Cell Proliferation], both filed by this inventor, placental alkaline phosphatase was shown to also enhance proliferation of human fibroblasts. In both applications which are incorporated herein by reference in their entirety, the effects of PALP alone were presented; however, its effects in the presence of Lf as described in the present application have never been reported.

In a third application by the inventor [Provisional U.S. patent application Ser. No. 11/430,574 filed May 9, 2006 and entitled ““Promoting Wound Healing and Skin Regeneration] PALP was used together with three other proteins as active components for the repair of tissue damage. In this last application, a composition containing only Lf and PALP as active components has not been provided.

As reported in earlier applications, full length PALP enzyme in its native state and exhibiting alkaline phosphatase activity is not required to achieve certain physiological effects such as promotion of proliferation and survival of fibroblasts in vitro. For example, both digestion of PALP with the protease bromelain and elimination of alkaline phosphatase activity through mutation provided active derivatives of PALP that were effective in vitro [U.S. patent application Ser. No. 10/653,622, filed Sep. 2, 2003 and entitled “Use of Placental Alkaline Phosphatase to Promote Skin Cell Proliferation”; U.S. Pub. App. 2005/0048046, published Mar. 3, 2005]. Therefore, one may expect that a smaller PALP-derived molecule devoid of alkaline phosphatase activity may be as active in vivo as the full-length PALP.

Consequently, active derivatives having similar number or less amino acid units than the full-length PALP may be suitable in the practice of embodiments of the invention so long as they exhibit physiological effects similar to that of native PALP. By way of example, modification of a PALP amino acid sequence or an active derivative, such as by exchanging one or more amino acids at critical positions, may enhance the biological activities of the resulting protein as related to the promotion of healing of wounded or unhealthy skin. Likewise, chemical or enzymatic changes in the level and position of glycosylation may maintain or enhance the effects of PALP or its derivatives. A smaller and still biologically active PALP can be produced by employing suitable proteolytic enzymes or by using appropriate methods of molecular biology to generate deletion mutants. The use of such altered or modified proteins is within the scope of embodiments of the present invention as much as the alterations or modifications remain limited and result in an active derivative that is a biologically functional equivalent.

Human PALP in solid form is available commercially from Sigma-Aldrich (St. Louis, Mo.), for example (Sigma catalog number P3895; CAS Registry Number 9001-78-9). Another commercial source of human PALP is Calbiochem (San Diego, Calif.; catalog number 524604).

For the experiments described in the Examples below, PALP was highly purified from commercial (Sigma-Aldrich) PALP preparation by a method essentially described earlier [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167]. In the experience of the inventor, in various PALP preparations purchased from Sigma-Aldrich, PALP represents up to 15% of the total protein.

Human PALP, and particularly a smaller molecular mass active derivative may also be obtained by chemical synthesis using conventional methods. For example, solid-phase synthesis techniques may be used to obtain PALP or an active derivative.

Recombinant methods of obtaining suitable preparations of PALP or active PALP derivatives are also suitable. Using the cDNA of PALP, recombinant protein may be produced by one of the many known methods for recombinant protein expression. PALP has been cloned and expressed in cells as described by Kozlenkov, et al. [Kozlenkov, A., Manes, T., Hoylaerts, M. F. and Millan, J. L. (2002), “Function assignment to conserved residues in mammalian alkaline phosphatases,” J. Biol. Chem. 277, 22992-22999]. Production of recombinant PALP by bacteria [Beck, R. and Burtscher, H. (1994), “Expression of human placental alkaline phosphatase in Escherichia coli,” Protein Expression and Purification 5, 192-197] and yeast [Heimo, H., Palmu, K. and Suominen, I. (1998), “Human placenta alkaline phosphatase: Expression in Pichia pastoris, purification and characterization of the enzyme,” Protein Expression and Purification 12, 85-92] is also a suitable method of obtaining PALP for use with embodiments of the present invention. However, experts in the art can also modify existing techniques to produce PALP or its active derivative by suitable mammalian cells, larger animals (rabbits, cows, pigs) or even plants.

Bacterial expression yields non-glycosylated PALP. So far there is no evidence that native glycosylated PALP and bacteria-produced PALP would have significantly different effects on cell proliferation or cell survival. Thus, in the embodiments of the present invention native glycosylated PALP and its active derivatives as well as non-glycosylated PALP and its active derivatives can be used interchangeably.

A PALP preparation that is commercially available contains impurities. Impure PALP preparations can be used as starting material to obtain homogeneous PALP by successive chromatographic steps, as described in detail in Example 5. Impure PALP preparations may also be used in formulating the compositions for use in the practice of embodiments of the present invention, so long as the given composition comprises a suitable amount of PALP, and impurities are not toxic and do not interfere with the beneficial effects of the components.

A preparation containing human PALP may also be obtained by extraction from placental tissue. Human placenta synthesizes the enzyme during pregnancy, so that toward the end of the third term the level of PALP in the placenta tissue and the maternal/fetal blood becomes very high. By way of example, a preparation may be obtained by butanol extraction of homogenized placenta. Other methods of extraction from placental tissue are also suitable.

Raw placental extracts that are not further enriched in PALP by using physical concentration methods cannot be expected to have physiological effects similar to those observed for the preparation of sufficiently enriched or purified or homogenous PALP, for at least two reasons. First, the relative concentration of PALP in an extract will be too low to expect a readily detectable effect in the skin. Second, raw placental extracts contain not only many different proteins but also other kinds of compounds, such as many lipids, proteolipids, carbohydrates, metals, vitamins, and the like.

Therefore, if placenta-derived PALP preparation is to be used in the embodiments of the present invention, a raw extract should be treated to enrich the concentration of PALP and obtain a purified preparation. A purified preparation will have a higher concentration of the active component than found in a raw tissue extract, such as a raw placental extract. The term “purified” is used herein to encompass compositions that are obtained from a starting material by one or more purification steps (such as solvent extraction, column separation, chromatographic separation, etc.) that enrich the concentration of PALP, relative to the starting material. The term “purified” should not be construed to connote absolute purity or homogeneity.

A further consideration in the practice of embodiments of the invention is the degree of purity that is required for the use in wound healing and skin care compositions. An advantage of using a preparation comprising highly purified or homogeneous PALP in the methods and treatment regiments of embodiments of the present invention is that possible side effects caused by contaminating proteins will not likely be an issue. However, impure PALP or PALP that is purified but not homogeneous also can be used in the compositions described herein, so long as no adverse effects are observed. Since each additional purification step results in significant loss of the protein, using a less pure PALP material for the compositions would be more cost-effective.

Compositions Comprising PALP and Lf

In one embodiment, the present invention provides a composition for promoting proliferation and/or survival of skin cells in the epidermis and dermis of wounded or healthy and unhealthy skin, comprising a physiologically compatible carrier and a therapeutically effective amount of a combination including both a) lactoferrin or active derivative thereof, and b) PALP or active derivative thereof. In another embodiment, the invention provides a composition for reducing inflammation or microbial load in the epidermis and dermis of a subject's skin.

The compositions provided by embodiments of the present invention have multiple uses. The compositions are suitable for treating non-chronic wounds, chronic wounds, transplanted skin, transplanted skin suspensions, unhealthy skin and healthy skin. Forms of wounds or skin damage that may be treated include surgical wounds (including wounds of the cornea caused by laser surgery or other kinds of surgeries), accidental (burned or cut) wounds, sunburns, or wounds generated by cosmetic surgeries. Other applications include increasing the efficacy of various skin grafting procedures and preventing or reversing skin damage induced by aging or environmental stresses. A particularly important application is to heal chronic wounds, such as diabetic ulcer, characterized by chronic inflammation as well as inadequate survival and proliferation of skin cells. Many additives and enhancing agents can be used to further promote the effects of these protein components, as described further below.

As discussed above, both Lf and PALP have separately been shown to positively affect wound healing. However, the two proteins have never been used together for skin treatment in any form prior to the present application. As used herein with respect to the present application, the phrase “active component” refers generally to Lf or active derivative thereof, or PALP or active derivative thereof.

Embodiments of the invention further include the use of Lf and PALP, or active derivatives thereof, in the manufacture of injectable compositions effective for promoting proliferation and/or survival of skin cells in addition to reducing inflammation or microbial load in the epidermis and dermis of wounded as well as healthy and unhealthy skin. Embodiments of the invention further include the use of Lf and PALP, or active derivatives thereof, in the manufacture of suitable topical compositions that effectively promote proliferation and/or survival of skin cells, or reduce inflammation or microbial load, in the epidermis and dermis of wounded as well as healthy and unhealthy skin.

The experiments described in the Examples below demonstrate that a topically applied composition containing both human Lf and human PALP is more effective in promoting healing of a burn wound, a well-reproducible human wound healing model, than compositions comprising either protein alone. By extension, it is expected that the positive combined effects of Lf and PALP on the healing of burn wounds can be applied with similar effects for many forms of skin injury and skin damage as well as for the prevention and reversal of stress-induced deterioration of skin's texture.

Compositions for Topical Application

For topical application an appropriate form of the composition is a cream (such as comprising vaseline or petrolatum). Other forms of the composition may include a gel, lotion, unguent, emollient, colloidal dispersion, suspension, emulsion, oil, spray, foam, mousse, and the like. Compositions suitable for topical application may also include, for example, liposomal carriers made up of lipids or special detergents.

For administration of the composition by topical application to a human, a physiologically compatible or acceptable carrier should be used. A carrier may be in any physical form appropriate for topical application to the skin. Any physiologically compatible carrier in which the active components are at least minimally soluble is suitable for topical compositions in embodiments of the present invention.

A physiologically acceptable carrier is one that is non-toxic, does not elicit an adverse physical reaction upon administration, and in which the active components are sufficiently soluble so that the composition can provide an effective amount of each of the active component. The carrier should also provide the composition an appropriate consistency for topical administration and should be capable of achieving proper distribution of the active component to the treated tissue. Preferably, no or minimal chemical reactions should take place between the carrier and the active components so that the lifetime of the latter is not appreciably reduced and no significant amounts of degradation products are formed.

Suitable carriers generally include, for example, water, acetone, ethanol, ethylene glycol, propylene glycol, butane-1,3-diol, isopropyl myristate, isopropyl palmitate, mineral oil, plant oil, gel-forming materials, cream-forming materials, liposomes made up of lipids or special detergents, mixtures thereof and the like. Buffered solutions can also serve as carriers.

In some embodiments, the topical composition is a gel. The gel may include as a carrier a gel-forming material that can be, but not limited to, water-soluble polymers and water swellable polymers. A representative, but incomplete, list of suitable carriers for a gel composition includes: calcium alginate, polyacrylic acid, polymethylacrylic acid, polyvinylpyrrolidine, polyvinyl alcohol, methylcellulose, sepharose, agar, agarose, dextran, starch, glycosaminoglycan polymers (i.e. hyaluronic acid, chondroitin, chondroitin-4-sulfate, chondroitin-6-sulfate, dermatan sulfate, keratan sulfate, heparin and heparin sulfate), pectin, dextranpolyethylene, chitosan, polyhydroxyethylmethacrylate, hydrophilic cellulose polymer, polyethylene glycol, amylose, polyethyleneoxide, collagen, gelatin, fibronectin, acrylamide polymer, or combination thereof.

By way of example, the active components can be incorporated into sterile 3% by weight methyl cellulose gel, 1% by weight agarose gel, 4% by weight gelatin gel, or 1 to 3% by weight calcium alginate. Gels of more complex compositions can be formulated. Those skilled in the art will recognize how to vary these components to obtain sustained release of active components of the invention.

In some embodiments, the carrier includes vaselinum flavum (yellow petrolatum), vaselinum album (white petrolatum), or vaselinum cholesterinatum. Commercially available vaselinum cholesterinatum consists of about 1.5 wt.-% cholesterol, about 5.0 wt.-% cerae lanae, and about 93.5 wt.-% vaselinum flavum.

The proteins may also be linked to nanoparticles and applied to the skin in an acceptable carrier as listed above. Nanoparticle-bound proteins are likely to assure a more localized and sustained response because they are less diffuse than proteins suspended in the above carriers in the absence of nanoparticles.

Purified protein or homogeneous protein preparations can be used to provide the active components in the compositions described herein. The less pure the protein preparations are, proportionally more of the protein preparations must be used for the given topical composition in order to provide effective amounts of the active components. Alternatively, preparations containing synthetic proteins or their active derivatives, or recombinant proteins or their active derivatives, can be employed as active components. Finally, impure protein preparations can be used to provide active components so long as the impurities do not cause significant side effects.

As indicated above, in addition to the carrier the composition comprises a therapeutically effective amount of the combination of active components. The phrase “therapeutically effective amount” in this context indicates a proportion and total concentration of active components that has greater effect in: (a) enhancing proliferation and reducing death of keratinocytes and/or fibroblasts or other cells in the epidermis and dermis, (b) promoting healing of wounds, (c) restoring or maintaining the strength or thickness of unhealthy and healthy skin, (d) decreasing the influx of inflammatory cells into the treated skin area and as a result decreasing the level of inflammation, or (e) decreasing the level of microbial load in the affected skin area; relative to a composition containing only one of the active components.

The required amount of an active component may vary based on the needs or tolerance of the individual subject, the degree to which the ability of keratinocytes and fibroblasts to proliferate has degenerated, the degree to which strength or thickness of the subject's skin has deteriorated, or other criteria evident to one of ordinary skill in the art.

Generally, the concentration of an active component in a composition for topical application will be at least about 0.01 wt.-%, and more suitably, from about 0.05 to about 1 wt.-%. In one embodiment, the composition comprises both active components and contains about 0.05 to about 0.5 wt.-% of each of the active components, with a total active protein content of not greater than about 1 wt.-%. As used herein, the phrase “active protein” refers to all forms of a protein (i.e., PALP or another alkaline phosphatase, and any active derivatives thereof, or Lf and any active derivatives thereof) present in a composition and having the desired physiological activity.

In some embodiments, the composition suitable for topical application consists essentially of the physiologically compatible carrier as a major ingredient, and the active component(s) as minor ingredients. By way of example, in one embodiment, the composition consists essentially of vaselinum cholesterinatum, lactoferrin or an active derivative thereof, and PALP or an active derivative thereof.

In other embodiments, the composition may include one or more additives or enhancers, such as preservatives, biologically active compounds with positive effects on skin cells, buffers, moisture-control compounds, or antibiotics, for example.

Additives or enhancers may be included in the topical compositions. The criterion for using an additive is that it increases, or at least does not significantly impair, the effectiveness of the active components in achieving the desired beneficial effect. Additives or enhancers in compositions for topical applications may include various ingredients, for example, preservatives (such as parabens, quaternary ammonium compounds, alcohols, phenols, essential oils and the like), buffers, antioxidants (such as vitamin E), antimicrobials, vitamins, nutrients (such as essential and non-essential amino acids, choline, inositol, minerals, trace metals, salts, nucleosides, purines, pyrimidines, monosaccharides, disaccharides, carbohydrates) and moisture-control agents (such as glycerine, propylene glycol, and the like). Other potential additives include, for example, analgesics, anesthetics, anti-acne agents, anti-dermatitis agents, anti-puritic agents, anti-inflammatory agents, anti-hyperkeratolytic agents, antiperspirants, anti-psoriatic agents, anti-seborrheic agents, anti-aging agents (such as retinoids including vitamin A), anti-wrinkle agents, anti-lightening agents, depigmenting agents, corticosteroids, additional tanning agents or hormones. Other additives may include, for example, colorants, sunscreens, emulsion stabilizers, preservatives, fragrance, humectants, waterproofing agents, viscosity modifying agents and the like.

One class of enhancers is represented by oxidants, such as hydrogen peroxide [Sen, C. K., Khanna, S., Babior, B. M., Hunt, T. K., Ellison, E. C. and Roy, S. (2002), “Oxidant-induced vascular endothelial growth factor expression in human keratinocytes and cutaneous wound healing,” J. Biol. Chem. 277, 33284-33290] and agents that act via oxidants such as, for example, grape seed proanthocyanidin extract [Khanna, S., Venojarvi, M., Roy, S., Sharma, N., Trikha, P., Bagchi, D., Bagchi, M. and Sen, C. K. (2002), “Dermal wound healing properties of redox-active grape seed proanthocyanidins,” Free Radical Biol. Med. 33, 1089-1096] and arginine, a substrate for nitric oxide generation [Shi, H. P., Efron, D. T., Most, D., Tantry, U.S. and Barbul, A. (2000), “Supplemental dietary arginine enhances wound healing in normal but not inducible nitric oxide synthase knockout mice,” Surgery 128, 374-378]. There are many other agents that can enhance the level oxidants in the skin.

Another class of enhancers is represented by agents that can further reduce inflammation such as, for example, estrogen [Ashcroft, G. S., Mills, S. J., Lei, K., Gibbons, L., Jeong, M.-J., Taniguchi, M., Burow, M., Horan, M. A., Wahl, S. M. and Nakayama, T. (2003), “Estrogen modulates cutaneous wound healing by downregulating macrophage migration inhibitory factor,” J. Clin. Invest. 111, 1309-1318], α₁-acid glycoprotein (also known as orosomucoid) [Fournier, T., Medjoubi-N, N. and Porquet, D. (2000), “Alpha-1-acid glycoprotein,” Biochim. Biophys. Acta 1482, 157-171; Vasson, M. P., Roch-Arveiller, M., Couderc, R., Baguet, J. C. and Raichvarg, D. (1994), “Effects of alpha-1 acid glycoprotein on human polymorphonuclear neutrophils: influence of glycan microheterogeneity,” Clinical Chimica Acta 224, 65-71; Williams, J. P., Weiser, M. R., Pechet, T. T. V., Kobzik, L., Moore, F. D. Jr. and Hechtman, H. B. (1997), “α₁-Acid glycoprotein reduces local and remote injuries after intestinal ischemia in the rat,” Am. J. Physiol. 273, G1031-G1035], and α₁-antitrypsin [Janciauskiene, S. (2001), “Conformational properties of serine proteinase inhibitors (serpins) confer multiple pathophysiological roles,” Biochim. Biophys. Acta 1535, 221-235]. α₁-Antitrypsin may also contribute to the effects of Lf and PALP through stimulating proliferation of skin fibroblasts [She, Q.-B., Mukherjee, J. J., Crilly, K. S. and Kiss, Z. (2000), “α₁-Antitrypsin can increase insulin-induced mitogenesis in various fibroblast and epithelial cell lines,” FEBS Lett. 473, 33-36; Dabbagh, K., Laurent, G. J., Shock, A., Leoni, P., Papakrivopoulou, J. and Chambers, R. C. (2001), “Alpha-1-antitrypsin stimulates fibroblast proliferation and procollagen production and activates classical MAP kinase signaling pathways,” J. Cell. Physiol. 186, 73-81].

Compounds in an additional class of enhancers are expected to promote the effects of Lf and PALP in vivo based on their positive effects on the proliferation and survival of cells in vitro. The non-limiting list includes: (a) ethanolamine, which is required for proliferation of keratinocytes as well as ethanolamine derivatives such as monomethylethanolamine and dimethylethanolamine, [Arthur, G. and Lu, X. (1993), The ethanolamine requirement of keratinocytes for growth is not due to defective synthesis of ethanolamine phosphoacylglycerols by the decarboxylation pathway,” Biochem, J. 293, 125-130], (b) zinc and calcium that are required for the optimal effects of PALP on cell proliferation [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167], (c) growth regulatory agents including progranulin [He, Z., Ong, C. H. P., Halper, J. and Bateman, A. (2003), “Progranulin is a mediator of the wound response,” Nature Medicine 9, 225-228], vasoactive intestinal polypeptide [Granoth, R., Fridkin, M and Gozes, I. (2000), “VIP and the potent analog, stearyl-Nle¹⁷-VIP, induce proliferation of keratinocytes,” FEBS Lett. 475, 78-83], angiopoietin-related growth factor [Oike, Y., Yasunaga, K., Ito, Y., Matsumoto, S.-I., Maekawa, H., Morisada, T., Arai, F., Nakagata, N., Takeya, M., Masuho, Y. and Suda, T. (2003), “Angiopoietin-related growth factor (AGF) promotes epidermal proliferation, remodeling, and regeneration,” Proc. Natl. Acad. Sci. USA 100, 9494-9499], galectins-3 and -7 [Cao, Z., Said, N., Amin, S., Wu, H. K., Bruce, A., Garate, M., Hsu, D. K., Kuwabara, I., Liu, F.-T. and Panjwani, N. (2002), “Galectins-3 and -7, but not galectin-1, play a role in re-epithelization of wound,” J. Biol. Chem. 277, 42299-42305], dermatan sulfate [Trobridge, J. M., Rudisill, J. A., Ron, D. and Gallo, R. L. (2002), “Dermatan sulfate binds and potentiates activity of keratinocyte growth factor (FGF-7),” J. Biol. Chem. 277, 42815-42820], transferrin [Carlevaro, M. F., Albini, A., Ribatti, D., Gentili, C., Benelli, R., Cermelli, S., Cancedda, R. and Cancedda, F. D. (1997), “Transferrin promotes endothelial cell migration and invasion: Implication in cartilage neovascularization.” J. Cell. Biol. 136, 1375-1384], prostaglandin, (d) growth factors including platelet-derived growth factor, transforming growth factor-α, transforming growth factor-β, epidermal growth factor, fibroblast growth factor, growth hormone, erythropoietin, hematopoietin, cytokines, insulin, insulin-like growth factor 1, vascular endothelial growth factor, nerve growth factor, granulocyte macrophage colony stimulating factor, placental growth factor, keratinocyte growth factor that are known to positively influence proliferation and survival of skin cells, and (e) wound repair promoters such as hyaluronic acid, fibronectin, laminin, collagen, elastin, tenasin, glycosamineglycans, proteoglycans, and integrins.

In certain embodiments, the composition for topical application includes a penetration-enhancing additive that enhances penetration of the active component(s) into the skin. Many conventional penetration enhancers are suitable in the practice of the invention. Non-limiting examples of suitable penetration enhancers include: sulfoxides such as dimethyl sulfoxide (DMSO); alcohols such as ethanol; polyols such as propylene glycol; surfactants such as sodium lauryl sulfate, lecithin, docusate sodium, and polysorbates; fatty acids such as lauric acid, myristic acid, palmitic acid, mineral oil, and stearic acid; esters such as isopropyl palmitate and isopropyl myristate; and amide such as urea.

Compositions for topical administration can be made using any number of suitable techniques. For example, a carrier, a protein preparation comprising one or more active components, and any optional additives and enhancers can be mixed together using a commercial mixer to form a suspension, gel, solution or the like. Conventional methods known in the art are suitable. The compositions can be additionally processed before and after formulation. Sterilization, for example, can be conducted individually by filter sterilization, while the whole composition can be irradiated, heat-treated or the like. Methods for conducting these steps are also conventional in the art.

A step of heat-activation of PALP or PALP-containing compositions may be included during the preparation of compositions. The stimulatory effects of PALP on fibroblast proliferation in vitro is enhanced by pre-heating it at 65-75° C. for 30 min [Q.-B. She, J. J. Mukherjee, J.-S. Huang, K. S. Crilly, and Z. Kiss (2000), “Growth factor-like effects of placental alkaline phosphatase in human fetus and mouse embryo fibroblasts,” FEBS Letters, 468, 163-167]. It is reasonable to expect that pre-heating of PALP prior to making the composition or pre-heating of the PALP-containing composition at 65-75° C. also will result in enhanced stimulation of skin cell proliferation in vivo.

Compositions for Injection

For injection of a composition comprising at least one active component, the carrier can be any physiologically acceptable carrier that does not cause an undesirable physiological effect and is capable of ensuring proper distribution of the active components in the treated tissue. One or more active component is dissolved or dispersed in the physiologically acceptable carrier.

Non-limiting examples of carriers include physiological saline and phosphate-buffered saline. Alternatively, the active component(s) may be enclosed in liposomes such as immunoliposomes, or other delivery systems or formulations that are known to the art may be employed. By way of example, the active component(s) can be readily dissolved in physiological saline (0.9 N NaCl), or in any other physiologically competent carrier, to yield a solution for injection.

The injectable compositions can be modified by any number of suitable additives and enhancers, such as many of those listed above for the topical application, that can be dissolved or suspended in the composition and that are expected to promote the effects of the active components or diminish any potential side effect.

An injectable composition can be prepared by dissolving or dispersing suitable preparations of the active component in the carrier using conventional methods. The proteins may also be linked to specifically formulated nanoparticles suspended in a suitable liquid carrier. As examples only, one suitable composition for the practice in the method comprises either Lf or PALP in a 0.9 N physiological salt solution to yield a total protein concentration of 10 mg/ml. Another suitable composition comprises both Lf and PALP in a 0.9 N physiological salt solution to yield a total protein concentration of 20 mg/ml.

A step of heat-activation of PALP or PALP-containing compositions may be included during the preparation of compositions.

Methods of Administration

Embodiments of the invention provides methods for promoting proliferation and/or survival of skin cells in the epidermis and dermis of wounded or non-wounded (unhealthy or healthy) skin of a subject. The methods may therefore be effective in restoring or maintaining the strength and thickness of wounded as well as unhealthy and healthy skin. In addition, the methods may also be effective to reduce inflammation or microbial load in the epidermis and dermis of the skin.

The methods described here are generally suitable for treatment of mammalian skin. The subject can be canine, porcine, horse, or bovine, for example. In one embodiment, the subject is human.

The compositions may be applied to wounded or non-wounded, unhealthy or healthy skin areas. Wounds suitable for treatment include injuries such as cut, puncture, abrasion, sore, scar, bruise, and burns of moderate- to high-degree that result in morphological and/or functional loss of continuity. Accidents, surgeries (including cosmetic surgeries, laser surgery, etc.), serious health conditions (for example, diabetes) and various cosmetic treatments may cause such wounds or injuries.

Unhealthy skin suitable for treatment, such as aging skin or skin exposed to excessive sunlight, is weaker, less elastic, and is more prone to injury than healthy skin. The structure of unhealthy or damaged skin is inferior to that of healthy skin (for example, the dermis and epidermis contain fewer cells and collagen). One purpose for treating unhealthy skin is to reduce further deterioration of skin and restore its function to normal or near-normal level.

Healthy skin is also suitable for treatment to prevent deterioration of skin induced by aging or environmental stress including excessive sunlight and microbial infection.

In one embodiment, the invention provides a method for promoting proliferation and/or survival of skin cells in the epidermis and dermis of wounded or non-wounded (unhealthy or healthy) skin, comprising the step of topically applying to the affected (wounded or damaged) or protected (healthy) area of the skin a composition comprising a therapeutically effective amount of a combination including: a) lactoferrin or active derivative thereof, and b) PALP or active derivative thereof. The composition may be applied both to the area of wounded or unhealthy skin or the surrounding healthy skin area.

In another embodiment, the invention includes a method for promoting proliferation and/or survival of skin cells in the epidermis and dermis of wounded or non-wounded (unhealthy or healthy) skin, comprising the step of injecting a composition including a physiologically acceptable carrier and a therapeutically effective amount of a combination of: a) lactoferrin or active derivative thereof, and b) PALP or active derivative thereof. For local administration, the composition may be injected into the wounded or non-wounded skin, and/or into healthy skin that surrounds the wounded skin, or both.

In yet another embodiment, the invention includes methods in which two active components are administered simultaneously or sequentially, but as separate compositions. For example, topical treatment of the wounded or non-wounded skin with either Lf, or PALP, or both, can be combined with intradermal or systemic applications of a complimentary composition. By way of example only, topical application of Lf can be combined with systemic application of PALP or Lf+PALP; topical application of PALP can be combined with systemic application of Lf, or Lf+PALP; topical application of Lf+PALP can be combined with systemic application of Lf, PALP, or Lf+PALP.

Such methods include the simultaneous or sequential performance of steps including:

administering to an area of the wounded or non-wounded skin either topically or by injection a first composition, wherein the first composition comprises at least one of Lf or active derivative thereof and PALP or active derivative thereof;

administering to an area of the wounded or damaged skin either topically or by injection a second composition, wherein the second composition comprises at least one of Lf or active derivative thereof and PALP or active derivative thereof;

wherein the first and second compositions comprise the same or different physiologically acceptable carrier;

wherein at least one of the first and second compositions comprises Lf or active derivative thereof;

and wherein at least one of the first and second compositions comprises PALP or active derivative thereof.

Administration by Topical Application

Topical administration can be accomplished via manual application of a composition such as a cream, a lotion, a gel or the like that includes one or more of the active components. A composition may be applied by other means, such as by spraying, applying with a pad or towel, etc. In some embodiments, a composition can be delivered by means of a dressing, bandage, patch, or other similar covering capable of releasing effective amounts of the active components. Other methods of delivering an active component are also within the scope of this invention.

Gels, creams, or other compositions for topical application may be applied directly to the affected site on the skin. In case of wounds, a gel or cream may optionally be applied in conjunction with a compliant porous or microporous substrate that provides a coating for the wound. A gel may have a desirable property over other forms, in that a gel may be able to absorb wound exudates while keeping the wound moist (due to the high water content).

In the practice of the methods described herein, topical application of the combination may be effective to stimulate proliferation and/or reduce the death of cells in the epidermis and dermis of the treated skin. Proliferation is stimulated when the rate of cell replication is increased, relative to the untreated condition, resulting in an increase in the number of skin cells. Increased cell survival also results in an increase in the number of skin cells because of decreased cell turnover. As demonstrated in the Examples, a visible sign of increased number of skin cells is more cell-rich dermis and increased number of epidermal cells and epidermal layers. Topical application may also be effective to decrease inflammation and microbial load in the treated skin area.

Topical treatment can be combined with other independent treatments including application of dressings (absorptives, alginates, hydrogels, hydrogel sheets, collagens, foams, elastic gauzes, hydrocolloids, silicone gel sheets, transparent films, wound fillers, and the like), enzymatic debridement (for example, Collagenase Santyl, and Acuzyme papain-urea), surgical debridement (physical removal of dead and ischemic tissue in a wound), cleansers (for example, Dermagran, Septicare, Hyperion, Biolex, Techni-care, UltraKlenz, Optipore Sponge, Gene Klenz, Medi Tech, Skintegrity, ClinsWound, Shur-Clens), oxygen therapy, hydrotherapy, compression (for example, Circular Boot, Flowplus, Flowpress, ArtAssist, EdemaFlow, high compression bandages, zinc-impregnated bandages, elastic bandages, leg wrappings, leg support), nutritional therapy (for example, high-protein and cholesterol-free supplements), cohesives, glues, sealants, and patches (for example, CryoSeal, Syvek, Quixil, Bio-Glue, Dermabond, CoSeal, Indermil, FocalSeal, LiquiBand).

As appropriate, topical treatment using the combination may be accompanied by simultaneous treatments with any of the available anti-diabetic medications.

Administration by Injection

In the practice of embodiments of the invention, injection may be employed as a mode of administration for a composition comprising one or more active component. In the case of injection, the active component(s) will be transported to the skin either directly (such as by intradermal application or partly subcutaneous application) or via the blood supply (such as by intravenous, intraperitoneal, intramuscular or subcutaneous applications). A composition comprising one or more active components may be administered via intravenous injection, intraperitoneal injection, subcutaneous injection, intradermal injection, intramuscular injection, or any other mode of delivery that ensures appropriate distribution and relative stability of the proteins in the body. If only one active component is injected, the other component is administered by another mode (such as topically).

The mode of injection is selected from intradermal, intravenous, subcutaneous, intramuscular, and intraperitoneal. The mode of injection is selected to provide either local delivery (such as intradermal application or partly subcutaneous application) or systemic delivery via the blood supply (such as intravenous, intraperitoneal and subcutaneous applications). For administration by injection, the composition(s) do not necessarily need to be injected into the wounded or unhealthy skin, but can be injected into healthy skin in close proximity to the wounded or unhealthy skin. Alternatively, for systemic delivery, the site of injection does not necessarily need to be in proximity to the wounded or unhealthy skin.

A common way to express a suitable dosage for systemic administration is grams of the active agent(s) per square meter of body surface area for the subject. Conventional formulas may be used to estimate a human subject's body surface area, based on the human's height (in cm) and mass (in kg).

In case of intravenous, intramuscular, intraperitoneal, or subcutaneous administration, the subject may be administered by injection a total of about 0.05 to 5 g active protein/m² per day. In another embodiment, a subject may be administered by intravenous, intramuscular, intraperitoneal, or subcutaneous application a total of about 0.05 to about 5 g active protein/m² twice or three times weekly. Alternatively, the subject may be administered a total of about 0.05 to about 5 g active protein/m² biweekly by intravenous, intramuscular, intraperitoneal, or subcutaneous administration.

In yet another embodiment, the subject may be administered a total of about 0.05 to about 5 g active protein/m² by intravenous, intramuscular, intraperitoneal, or subcutaneous application once daily for several days, with treatment then continued with less frequent applications of smaller doses.

One suitable treatment for damaged skin is by intradermal injection of the chosen composition(s). For one injection site, the subject may be administered a total of about 0.01 to 1 mg of active protein. Intradermal delivery of the composition can be performed once or twice daily, two-to-three times a week, once a week, or biweekly, as suitable.

If the chosen composition is injected locally, such as when the mode of injection is intradermal, aliquots of about 10 to 100 μL per injection site may be administered. The concentration of active protein in the injectable composition may be in the range of about 0.1 to 50 mg/mL. Alternatively, the concentration of the active protein may be in the range of about 0.5 to about 20 mg/mL. In one embodiment, a plurality of injection sites is treated for one administration.

Intradermal application may be especially effective and economical mode of application in the practice of the present invention. Intradermal application may require the use of less active protein as compared to other modes of injection. Also, for localized application the active components may be more effectively delivered or transported to the epidermal and dermal layers of the treated skin.

As appropriate, for the treatment of chronic wounds administration by injection may be accompanied by simultaneous treatments with any of the available anti-diabetic medications.

Application to Transplanted Skin

The methods described above are also suitable for promoting proliferation and/or survival of skin cells in the epidermis and dermis of transplanted skin. The methods may therefore be effective in restoring or maintaining the strength and thickness of transplanted skin. The methods may also be effective to reduce inflammation or microbial infection in a transplant site. Ultimately the method may be effective to enhance survival and proper functioning of the transplanted skin as well as promote the growth of the host skin (i.e. to increase the likelihood of a successful transplant).

In the practice of embodiments of the invention, the transplanted skin may be mammalian skin such as human skin, for example. In one embodiment the transplanted skin is intact human skin or human skin cell suspension (transplant). The transplanted skin may be transplanted onto a host in some embodiments, such as a human host. For the purposes of the present application, skin that is transplanted onto a host is considered to be “unhealthy” or “damaged” until completely healed. Therefore, all methods and compositions described herein for the treatment of unhealthy and damaged skin are likewise suitable for the treatment of transplanted skin.

The methods include a step of administering a combination of active components to transplanted skin by injection or topical application, for example. The methods may further comprise a step of administering the composition to an area of host skin adjacent to the transplanted skin.

For injection, the composition(s) do not necessarily need to be injected into the transplanted skin, but can be injected into host skin in close proximity to the wounded or damaged skin. Alternatively, for systemic delivery, the site of injection does not necessarily need to be in proximity to the transplanted skin.

The invention therefore provides in one embodiment a method for promoting proliferation and/or survival of skin cells in the epidermis and dermis of transplanted skin, comprising the step of topically applying to an area of the transplanted skin a composition comprising a therapeutically effective amount of a combination including: a) Lf or active derivative thereof, and b) PALP or active derivative thereof. The composition may be additionally applied to host skin that surrounds the transplanted skin.

In another embodiment, the invention includes a method for promoting proliferation and/or survival of skin cells in the epidermis and dermis of transplanted skin, comprising the step of injecting a composition including a physiologically acceptable carrier and a therapeutically effective amount of a combination of: a) Lf or active derivative thereof, and b) PALP or active derivative thereof. For local administration the composition may be injected into the transplanted skin, or into host skin that surrounds the transplanted skin, or both.

In yet another embodiment, the invention includes methods in which two active components are administered simultaneously or sequentially, but as separate compositions. For example, both the host skin and transplanted skin can be treated by combining topical applications and administration by injection to administer the active component(s) to the affected areas of host skin and transplanted skin. By way of example only, topical application of Lf can be combined with systemic application of PALP or Lf+PALP; topical application of PALP can be combined with systemic application of Lf, or Lf+PALP; topical application of Lf+PALP can be combined with systemic application of Lf, PALP, or Lf+PALP.

Such methods include the simultaneous or sequential performance of steps including:

administering to an area of transplanted skin or host skin either topically or by injection a first composition, wherein the first composition comprises at least one of Lf or active derivative thereof and PALP or active derivative thereof;

administering to an area of transplanted skin or host skin either topically or by injection a second composition, wherein the second composition comprises at least one of Lf or active derivative thereof and PALP or active derivative thereof;

wherein the first and second compositions comprise the same or different physiologically acceptable carrier;

wherein at least one of the first and second compositions comprises Lf or active derivative thereof; and

wherein at least one of the first and second compositions comprises PALP or active derivative thereof.

Another method of delivery of proteins is by incorporating protein-containing implants (such as, for example, small permeable capsules, a mini osmotic pump, or permeable vials) under the skin near the damaged area. By selecting the content and pore size of the permeable capsules and vials, it is possible to regulate the rate of outward diffusion of proteins. This method is known in the art.

Regimens for Treating Skin

The invention also provides regimens for treating wounded or unhealthy or healthy skin. The regimens include repeated administration of one or more composition as described above.

The combination is applied periodically over a period of time. As used with respect to the regimens described herein, the term “periodically” refers to repeated administration of the combination targeted to restoring or maintaining the strength and the thickness of the wounded or damaged skin over the time of treatment. The term “periodically” includes repeated administration at regular intervals, but also includes repeated administration over irregular intervals as determined by the subject's condition. For example, a regimen can include applications made on an “as needed” basis so long as administration is made repeatedly over a period of time.

The frequency of administration of the combination can vary and depends on the type of skin, the extent of injury or wound-free damage, the location of the treated skin, the concentration of the active components in the composition(s), and the method used to administer the composition(s).

In one embodiment, the invention includes a regimen for promoting proliferation and/or survival of skin cells in the epidermis and dermis of wounded or non-wounded skin, the regimen comprising periodically administering to the skin either topically or by injection a therapeutically effective amount of a combination including: a) Lf or active derivative thereof; and b) PALP or active derivative thereof.

Generally, a therapeutically effective amount of the combination is administered at each dosage. In the regimens of the invention, however, the amount of active protein that is administered does not need to be identical for each separate administration. More or less of one or both active components may be administered in separate administrations, as the subject's needs dictate. A medical professional supervising treatment can adjust administered dose to obtain the desired results.

The frequency of application and the duration of the regimen will depend on the size and nature (chronic, non-chronic) of the wound, transplant, skin damage, the area that needs protection from environmental stress, the extent of infection, the physiological state of the skin, the magnitude of response, and the level of satisfaction by the treated subject.

In one embodiment of the regimen, the combination is administered once or twice every day. In another embodiment of the regimen, the combination is administered twice or three times a week. In yet another embodiment, the combination is administered once a week.

A recommended treatment schedule for unhealthy skin or healthy skin that is exposed to stress includes administration of the combination twice weekly for several months or for a time period as required to obtain the desired result. A recommended treatment schedule for wounds is twice or three times per week. Variations to this treatment schedule will depend on the level of deterioration of skin and the level of success. For example, a regimen in which the combination is administered about once per day during the first week, about two to three times total during the second week, and about once per week thereafter may be suitable in the treatment of wounds particularly if accompanied with infection.

This invention may take on various modifications and alterations without departing from the spirit and scope thereof. Accordingly, it is to be understood that this invention is not to be limited to the above-described disclosures. It is also to be understood that this invention may be suitably practiced in the absence of any element not specifically disclosed herein.

In describing embodiments of the invention, specific terminology is used for the sake of clarity. The invention, however, is not intended to be limited to the specific terms so selected, and it is to be understood that each term so selected includes all technical equivalents that operate similarly.

### EXAMPLES

The experiments described below were designed to provide maximum reproducibility in the size and quality of wounds induced for experimental purposes. An area of dorsal skin including the transplanted human skin was burned by immersing the back of the mouse into 65° C. hot water for exactly 12 seconds by making sure that the water always had the same depth so that a similar size of skin was always immersed.

Another objective was to make the experiments as relevant as possible to humans. With these goals in mind, human skin transplanted into the dorsal skin of mice was used in the experiments. The experiments described below demonstrate that a topically applied composition containing both human Lf and human PALP is more effective in promoting healing of a burn wound than application of either protein alone.

### Examples 1-4

### Promoting Effects of Lf and PALP Alone or in Combination on the Healing of Epithelium and Dermis in Wounded Skin

These Examples demonstrate that in human skin transplanted onto the back of mice, topically applied Lf and PALP in combination enhance the restoration of epidermis and dermis in the wounded skin more efficiently than either protein alone.

In all experiments, seriously compromised immune deficient (“SCID”) mice were used. The mice were housed and handled under specific pathogen-free (“SPF”) conditions. They were used at 8-14 weeks of age.

Most of the subsequent experimental procedures, outlined below, were described in detail by Juhasz et al. [Juhasz, I., Simon, Jr., M., Herlin, M. and Hunyadi, J. (1996), “Repopulation of Langerhans cells during wound healing in an experimental human skin/SCID mouse model,” Immunology Letters 52, 125-128]. The histological analysis by hematoxylin/eosin is a conventional procedure that has been described, for example, by Wankell et al. [Wankell, M., Munz, B., Hubner, G., Hans, W., Wolf, E., Goppelt, A. and Werner, S. (2001), “Impaired wound healing in transgenic mice overexpressing the activin antagonist follistatin in the epidermis,” The EMBO J. 20, 5361-5372].

In all experiments, the treated skin was human skin that had been grafted onto the mouse skin. Children at ages between 0.5-6 years donated human foreskin. For the grafting procedure, a mouse (at age 6-8 weeks) was anesthetized with inhalation anesthetic methoxyflurane. SCID mice were utilized to minimize the risk of rejection of transplanted tissue by the host mouse. A circular graft bed of approximately 1.5 cm² was prepared on the lateral abdomen of the mouse by removing skin down to the fascia. The full-thickness donor foreskin was placed onto the wound bed and held in place with 5-0 monofilament sutures. The transplantation site was covered with a layer of non-stick gauze (CUTICERIN, available from Beiersdorf of Hamburg, Germany) and the adhesive bandage soaked with physiological saline and sutured to the dorsal and ventral skin of the animal with a surgical stitch. An additional layer of surgical tape was then applied. After 4-6 weeks of the surgery, the entire area of the transplanted skin was immersed into 65° C. water bath for exactly 12 seconds that caused second degree burns. The entire human skin area was treated with one of the creams within two minutes of the burning procedure. In each case, on the third day the bandage was removed and the treatment repeated.

Each time, an area of approximately 1.5 cm² was treated with 150 mg of various creams composed of one or two active components selected from homogeneous placenta-derived PALP and highly purified commercial Lf mixed in vaselinum cholesterinatum.

The following creams were prepared, in each case the indicated amount of active component(s) being added to 1-g vaselinum cholesterinatum: Lf cream, 1.2 mg Lf; PALP cream 1.2 mg PALP; Lf/PALP cream, 1.2 mg Lf+1.2 mg PALP. An additional cream (control cream) consisted only of vaselinum cholesterinatum.

For histology, skin samples were excised from the treated areas after six full days of treatment. The excised skin samples were fixed in 4% paraformaldehyde in phosphate-buffered saline and embedded in paraffin so that several consecutive cross-sections could be made. Skin sections (6 μm) were stained with hematoxylin/eosin (“H&E”). At least 15 skin sections, derived from 3-5 animals were evaluated for the effectiveness of each type of treatment.

In the following, pictures that give the best representation of findings are shown. However, it is important to note that, for example in case of burning, regeneration of skin does not take place evenly over the whole surface. New epithelium can be formed only from epithelial cells that survive burning, and these surviving cells are not evenly distributed. Accordingly, the extent of regeneration after six days of treatment will vary to some extent. For each treatment, two or three pictures were selected to document the spectrum of changes that were taken place in the epidermis and dermis. Comparison of the effects of various treatments on the thickness of epithelium (measured with an appropriate scale) revealed that combined treatment with Lf and PALP enhanced the thickness of epithelium about 2- to 3-times more than treatments with PALP or Lf alone.

### Example 1

### Effects of the Control Cream on the Epidermis and Dermis of Burned Human Skin

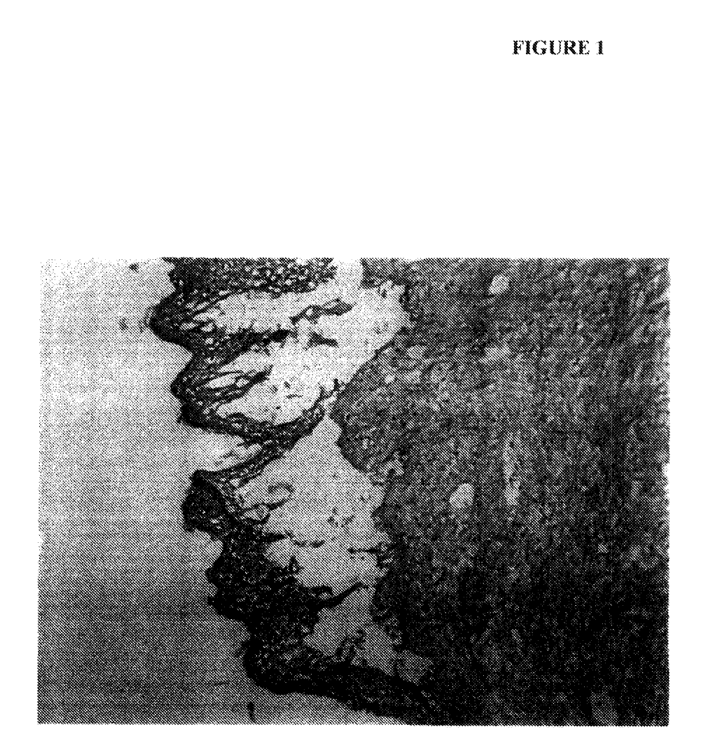

Within 2 minutes of burning the skin, the affected area was treated with the control cream (vaselinum cholesteratum) alone. FIG. 1 is a representative image of a histological sample taken from transplanted human skin six full days after the start of treatment. Segments of skin are separated from each other with no sign of any significant regeneration of the epidermis.

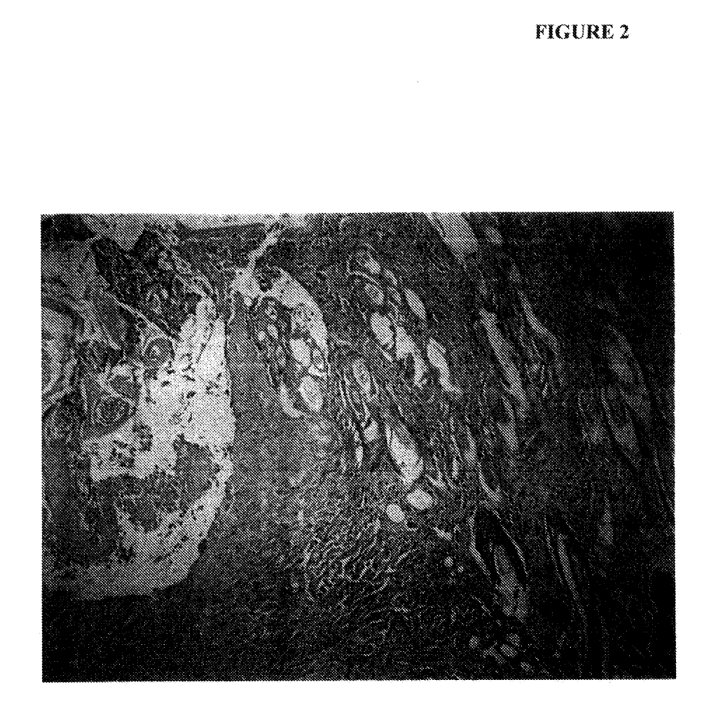

FIG. 2 is another representative image of a histological sample taken from transplanted human skin six full days after the start of treatment with the control cream. Again, there is no sign of epidermis formation, while the dermis also has a clearly unhealthy appearance with large a-cellular regions.

### Example 2

### Effects of the Lf Cream on the Epidermis and Dermis of Burned Human Skin

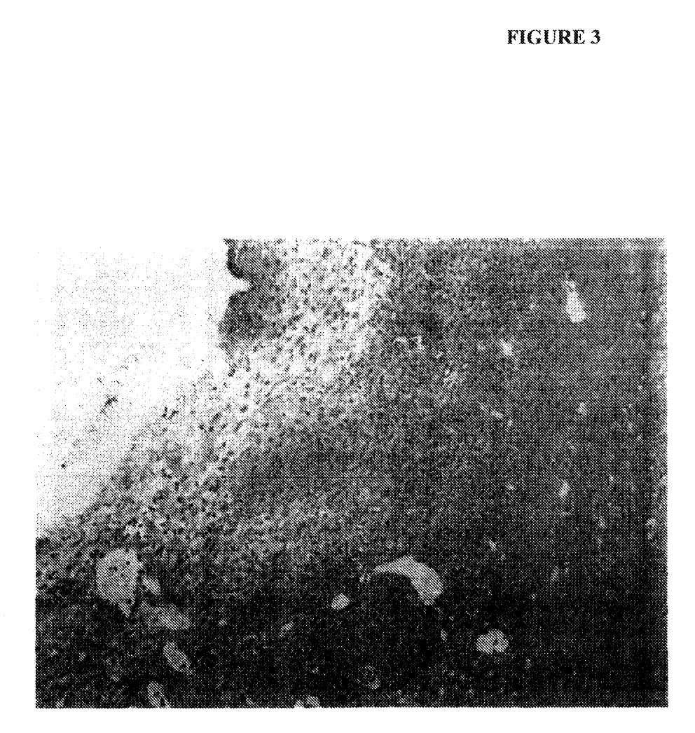

Within 2 minutes of burning the skin, the affected area was treated with the Lf cream. FIG. 3 is a representative image of a histological sample taken from transplanted human skin six full days after the start of treatment. While in this segment no newly formed epidermal cells or epidermal layer is found, the dermis is healthier with a higher level of cell density than seen in the samples from Example 1.

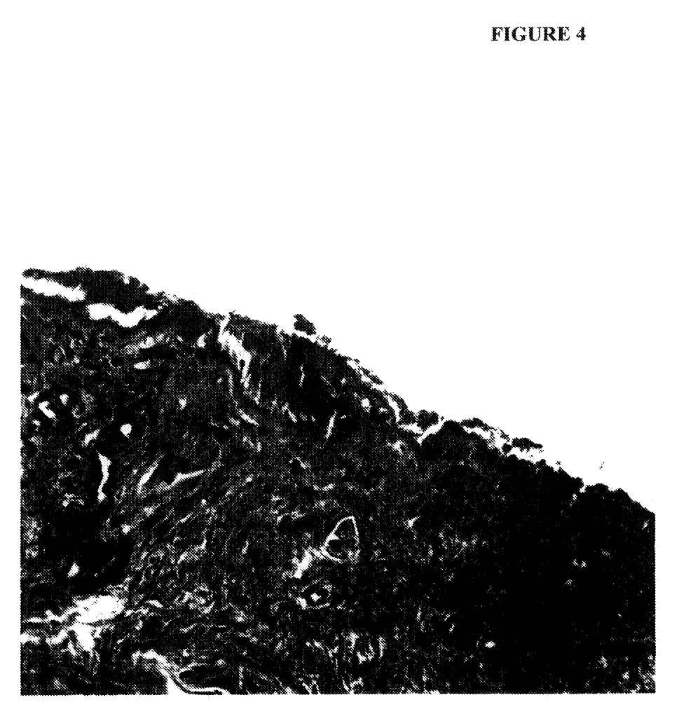

FIG. 4 is another representative image of a histological sample taken from transplanted human skin six full days after the start of treatment. In this skin section, a low-level regeneration of epidermis is seen as indicated by the blue color (represented by dividing epidermal cells) and the columnar structure of newly forming epidermis in the upper-center part of the picture.

Overall, these pictures confirm previous descriptions that Lf has positive effects on the regeneration of wounded skin, although the data also indicate that these effects are far from being robust.

### Example 3

### Effects of the PALP Cream on the Epidermis and Dermis of Burned Human Skin

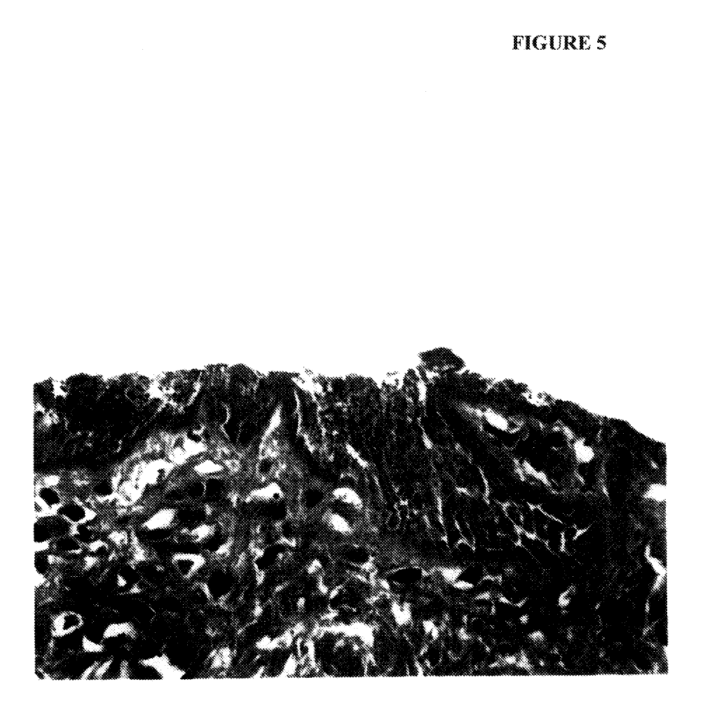

Within 2 minutes of burning the skin, the affected area was treated with the PALP cream. FIG. 5 is a representative image of a histological sample taken from transplanted human skin six full days after the start of treatment. In this skin section, a medium level regeneration of epidermis is seen as indicated by the blue color (representing dividing epidermal cells) and the well-developed columnar structure of newly forming epidermis in the upper section of the picture.

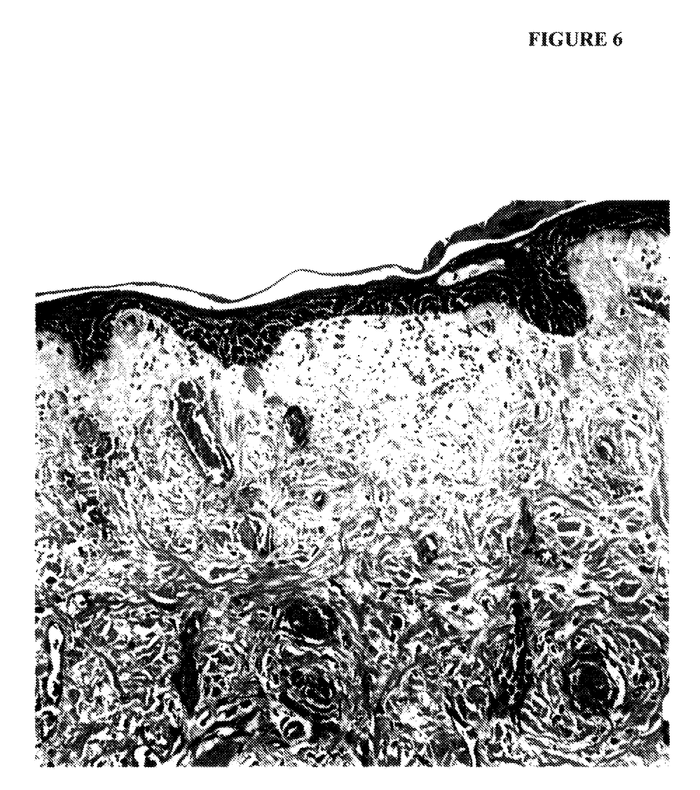

FIG. 6 is another representative image of a histological sample taken from transplanted human skin 6 full days after the start of treatment with the PALP cream. In this skin section again, a medium-level regeneration of epidermis is seen represented by 2-3 layers of epidermal cells (seen in the upper section of the picture).

In both representative samples the dermis was healthier containing more cells than the dermis in the Example 1 samples. Overall, PALP alone also had greater effects on the formation of epidermal cells and epidermal layers than Lf did.

### Example 4

### Effects of the Lf and PALP Cream on the Epidermis and Dermis of Burned Human Skin

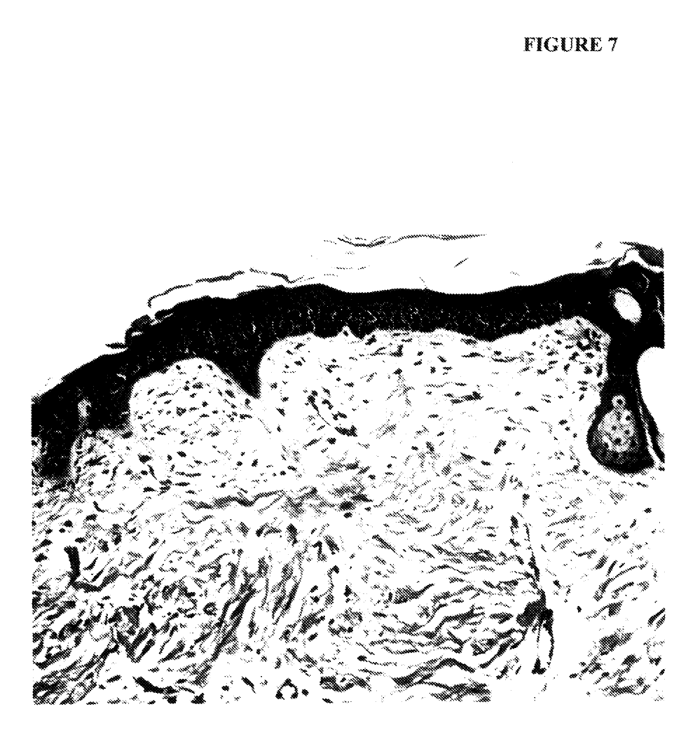

Within 2 minutes of burning the skin, the affected area was treated with a cream containing both Lf and PALP (Lf/PALP cream). FIG. 7 is a representative image of a histological sample taken from transplanted human skin six full days after the start of treatment. In this section, the newly formed epidermis is clearly thicker (on average consisting of 5 layers) than the epidermis formed either in the presence of Lf or PALP alone, although the dermis did not seem to contain more cells.

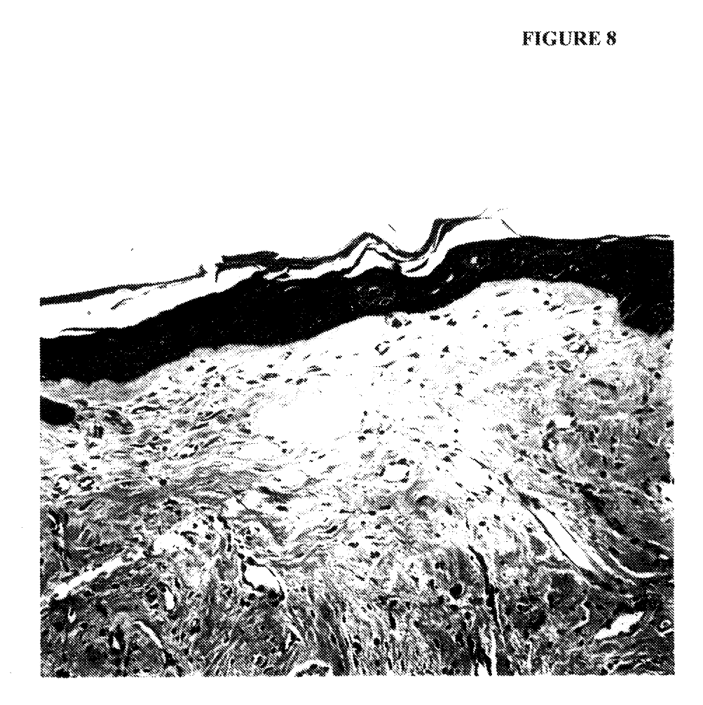

FIG. 8 is another representative image of a histological sample taken from transplanted human skin six full days after the start of treatment with the Lf/PALP cream. In this skin section again a higher level regeneration of epidermis is seen as represented on average by 4-6 layers of epidermal cells. As the result of treatment, cells densely populate the dermis.

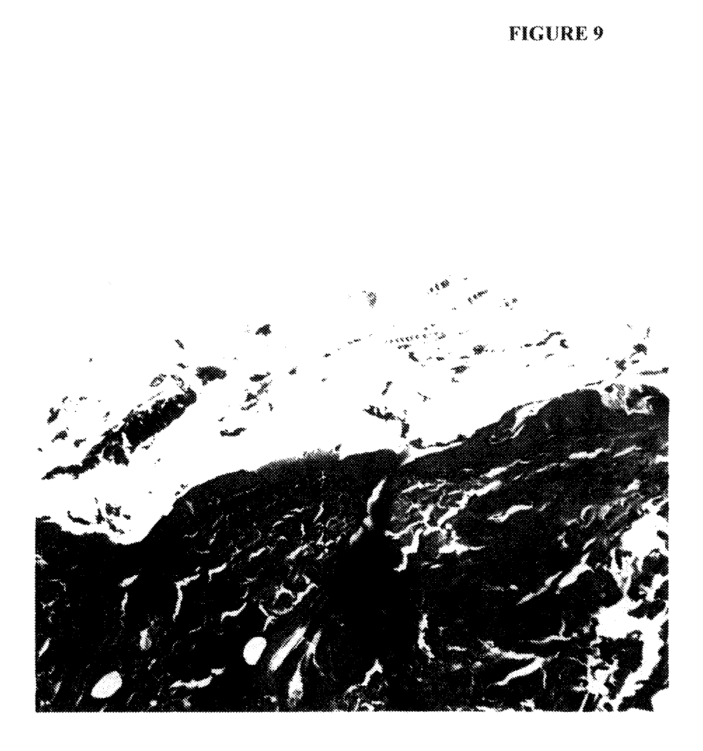

FIG. 9 is yet another representative image of a histological sample taken from transplanted human skin six full days after the start of treatment with the Lf/PALP cream. A higher level regeneration of epidermis can be seen as indicated by the reconstructed continuous columnar structure of epidermis. Again, cells densely populate the dermis. Overall, the combined treatment with Lf and PALP on average enhanced the thickness of epidermis about 2-fold or more compared to the treatments with either protein alone. Co-treatment with Lf and PALP also resulted in a dermis densely populated by cells, mostly fibroblasts.

### Example 5

### Purification and Spectrophotometric Assay of PALP

Human PALP (Type XXIV, 1020 units of total activity) in a partially purified form was obtained commercially from Sigma-Aldrich. Extraction of placental tissue with n-butanol, followed by one or more chromatographic steps, was performed by Sigma-Aldrich to obtain the partially purified material. Butanol extraction inactivates most of the other placental proteins, including growth factors, but does not reduce the mitogenic or enzymatic activity of PALP.

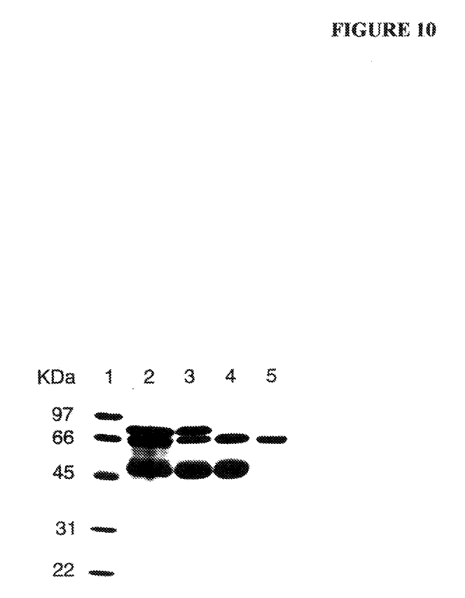

As determined by sodium dodecyl sulfate-polyacrylamide gel electrophoresis (SDS-PAGE), the partially purified PALP obtained from Sigma-Aldrich (denoted “commercial PALP” herein) was not homogeneous and contained other proteins. FIG. 10 shows the picture of a gel separation of a preparation comprising commercial PALP without further purification, and other preparations of PALP of increasing purity. Separation of proteins was performed by conventional SDS-PAGE, and proteins were stained with coomassie blue stain. Lane 1 contains various molecular mass standards for comparison. Lane 2 represents a preparation containing commercial PALP with a strong 52 kDa band representing α₁-antitrypsin and another strong 66 kDa band representing a mixture of PALP and albumin. Lanes 3 and 4 represent preparations comprising commercial PALP material after further purification steps (described below), and lane 5 represents a preparation of homogeneous PALP obtained by the complete purification procedure described below.

A purification procedure was performed to further purify the commercially obtained PALP to homogeneity. A slightly modified procedure described earlier [She, Q.-B., Mukherjee, J. J., Huang, J.-S., Crilly, K. S. and Kiss, Z. (2000), “Growth factor-like effects of placental alkaline phosphatase in human and mouse embryo fibroblasts,” FEBS Lett. 469, 163-167] was used which is incorporated by reference.

A solution of commercial PALP was prepared by dissolving 350 mg of commercial PALP into 10 ml of buffer A (0.1 M sodium acetate, 0.5 M NaCl, 1 mM MgCl₂, 1 mM CaCl₂, adjusted to pH 6.5). This solution was then further purified by successive Concanavalin A-Sepharose and Q-Sepharose chromatography, essentially following the procedure described elsewhere [Chang, T.-C., Huang, S.-M., Huang, T.-M. and Chang, G.-G. (1992), “Human placenta alkaline phosphatase: An improved purification procedure and kinetic studies,” Eur. J. Biochem. 209, 241-247].

The solution was run through a Concanavalin A-Sepharose column followed by an elution step using buffer A as solvent. For elution, buffer A included 50 mM α-methyl-D-mannopyranoside. The active fractions collected from the effluent were pooled and dialyzed against buffer B (50 mM Tris-HCl at pH 7.7). SDS-PAGE separation of the collected and dialyzed fraction is shown in FIG. 10 in lane 3.

The collected and dialyzed fraction from the previous step was then passed through a Q-Sepharose column. The fraction of interest was eluted with buffer B using a linear gradient of 0-250 mM potassium phosphate at a pH of 7.5. The active fractions from the Q-Sepharose column were pooled and dialyzed against phosphate-buffered saline and concentrated by Amicon ultrafiltration. SDS-PAGE separation of the collected and dialyzed fraction is shown in FIG. 10 in lane 4, which demonstrates that at least two major proteins are still present in the fraction after dialysis.

Then, the collected and dialyzed fraction from the previous step was purified to homogeneity by t-butyl hydrophobic interaction chromatography (HIC) as described above [She, Q.-B., Mukherjee, J. J., Huang, J.-S., Crilly, K. S. and Kiss, Z. (2000), “Growth factor-like effects of placental alkaline phosphatase in human and mouse embryo fibroblasts,” FEBS Lett. 469, 163-167]. Prior to adding the fraction to the t-butyl HIC column, the fraction was made 2 M in ammonium sulfate, and pH was adjusted to 6.8. The 5 ml bed volume t-butyl HIC cartridge (BIO-RAD, Hercules, Calif.) was connected to a fast performance liquid chromatography (FPLC) system from PHARMACIA (Peapack, N.J.). The fraction was introduced to the HIC column, and the column was eluted with buffer C (100 mM sodium phosphate buffer, 2 M ammonium sulfate at pH 6.8). The column was eluted with buffer C until a first protein-containing fraction completely eluted, and then a negative gradient of 2 M-to-0 M ammonium sulfate in 100 mM sodium phosphate at pH 6.8 was passed over the column. The negative linear gradient was used to elute a second protein-containing fraction, which contained the enzymatically active PALP protein. Note that this HIC chromatography step was not included in the procedure described by Chang et al [Chang, T.-C., Huang, S.-M., Huang, T.-M. and Chang, G.-G. (1992), “Human placenta alkaline phosphatase: An improved purification procedure and kinetic studies,” Eur. J. Biochem. 209, 241-247].

The enzymatically active fraction from the HIC separation was dialyzed against phosphate buffered saline and concentrated by Amicon ultrafiltration. The presence and purity of the PALP enzyme in the fraction was confirmed by SDS PAGE. After electrophoretic separation, the gel was stained using coomassie blue or silver stain for visual observation of protein bands. In many cases, a single protein band was observed with an approximate molecular weight of 66 kDa. The pure PALP was further identified by sequence analysis performed by the Mayo Clinic Protein Core Facility (Rochester, Minn., USA). Whenever the gel staining procedure still suggested the presence of a minor contaminant (which occurred in about 50% of cases), the HIC chromatography step was repeated. Invariably, after the second HIC chromatography step, gel staining did not suggest the presence of any contaminant protein.

PALP enzyme activity was assayed using a spectroscopic method by monitoring the hydrolysis of 4-nitrophenylphosphate (as an increase in absorbance at 410 nm) at room temperature (22° C.) as described elsewhere [Chang, G.-G., Shiao, M.-S., Lee, K.-R. and Wu, J.-J. (1990), “Modification of human placental alkaline phosphatase by periodate-oxidized 1,N⁶-ethenoadenosine monophosphate,” Biochem. J. 272, 683-690]. Activity analysis of 5-10 μg purified enzyme was performed in 1 mL incubation volume containing 50 mM Na₂CO₃/NaHCO₃, 10 mM MgCl₂, 10 mM 4-nitrophenylphosphate at pH 9.8. The extinction coefficient of 4-nitrophenol was taken as 1.62×10⁴ M⁻¹ cm⁻¹. An enzyme activity of 1 U (unit) is defined as 1 μmol substrate hydrolyzed/min at 22° C. at pH 9.8.

1. A composition for promoting proliferation and/or survival of skin cells in the epidermis and dermis of wounded or non-wounded, healthy or non-healthy skin or for reducing inflammation or microbial infection in the epidermis and dermis of wounded or non-wounded, healthy or non-healthy skin, comprising a combination of lactoferrin or an active derivative thereof and alkaline phosphatase or an active derivative thereof.

2. The composition of claim 1, wherein the alkaline phosphatase is placental alkaline phosphatase.

3. The composition of claim 1, wherein alkaline phosphatase is intestinal alkaline phosphatase, tissue non-specific alkaline phosphatase, or germ-cell alkaline phosphatase.

4. The composition of claim 1, wherein the alkaline phosphatase or active derivative is a human protein.

5. The composition of claim 1, wherein the alkaline phosphatase or an active derivative is a recombinant protein.

6. The composition of claim 1, wherein the lactoferrin or active derivative is a human protein.

7. The composition of claim 1, wherein the lactoferrin or an active derivative is a recombinant protein.

8. The composition of claim 1, further comprising a physiologically compatible carrier.

9. A method for promoting proliferation and/or survival of skin cells in the epidermis and dermis of wounded or non-wounded, healthy or non-healthy skin or for reducing inflammation or microbial infection in the epidermis and dermis of wounded or non-wounded, healthy or non-healthy skin comprising the step of administering a therapeutically effective amount of a combination comprising lactoferrin or an active derivative thereof and alkaline phosphatase or an active derivative thereof.

10. The method of claim 9, wherein the skin is human skin.

11. The method of claim 9, wherein the skin is transplanted skin.

12. The method of claim 9, wherein the lactoferrin is administered simultaneously or sequentially with alkaline phosphate.

13. The method of claim 12, wherein the step of administering comprises topically administering the combination to the skin.

14. The method of claim 13, wherein the composition is in the form of a cream, a gel, a lotion, an unguent, an emollient, a colloidal dispersion, a suspension, an emulsion, an oil, a spray, a foam, or a mousse.

15. The method of claim 13, wherein the topically administered composition comprises:
about 0.01 to about 0.5 wt.-% of lactoferrin or an active derivative thereof; and about 0.01 to about 0.5 wt.-% of alkaline phosphatase or an active derivative thereof.

16. The method of claim 13, wherein the topically administered composition has a total active protein content of not greater than about 1 wt.-%.

17. The method of claim 12, wherein the step of administering comprises injecting into the skin the combination in a physiologically acceptable carrier.

18. The composition of claim 17 wherein the carrier comprises physiological saline solution.

19. The method of claim 17, wherein the injection is intravenous, subcutaneous, intramuscular, or intraperitoneal.

20. The method of claim 19, wherein the total amount of active protein is about 0.01 gram to about 5 gram per square meter of calculated surface area for the mammal.

21. The method of claim 17, wherein the injection is intradermal.

22. The method of claim 21, wherein the total amount of active protein injected is about 0.01 mg to about 1 mg per injection site.

23. A method for promoting proliferation or survival of skin cells in the transplant skin cell suspensions, comprising the step of adding to the skin cell suspension a composition comprising a therapeutically effective amount of a combination comprising lactoferrin and alkaline phosphatase or active derivatives thereof.
